(19) United States
(12) Patent Application Publication
(10) Pub. No.: US 2015/0327847 A1
(43) Pub. Date: Nov. 19, 2015

(54) Sternum Spreader

(71) Applicant: Fehling Medical Corporation, Acworth, GA (US)

(72) Inventor: Guido Fehling, Karlstein (DE)

(21) Appl. No.: 14/580,607

(22) Filed: Dec. 23, 2014

(30) Foreign Application Priority Data
Dec. 30, 2013 (DE) 20 2013 105 963.3

(51) Int. Cl. A61B 17/02

(52) U.S. Cl. CPC A61B 17/0206

(57) ABSTRACT

The invention relates to a sternum spreader comprising two sternum blades, which are arranged in such a way that they can be moved in relation to each other, and each of which has a contact surface, wherein the sternum spreader comprises an elongated variable-length element with end faces, on which the sternum blades are disposed.

### CROSS REFERENCE TO RELATED APPLICATIONS

This patent application claims priority to German Patent Application 10 2014 101 573.8, filed on Feb. 7, 2014.

### STATEMENT REGARDING FEDERALLY SPONSORED RESEARCH OR DEVELOPMENT

No federa1 government funds were used in researching or developing this invention.

NAMES OF PARTIES TO A JOINT RESEARCH AGREEMENT

Not applicable.

### SEQUENCE LISTING INCLUDED AND INCORPORATED BY REFERENCE HEREIN

Not applicable.

### BACKGROUND

1. Field of the Invention

The present invention relates to a sternum spreader.

2. Background of the Invention

Sternum spreaders or retractors comprising two sternum blades, which are arranged in such a way that they can be moved in relation to each other, and each of which has a contact surface, are known from the prior art. In these known sternum retractors each of the sternum blades usually has an arm, by means of which the sternum blade is disposed on a sliding mechanism. Known sternum retractors are disclosed, for example, in the U.S. Pat. No. 5,520,610 or 5,088,472.

After the operation the sternum refractor is left in the body of the patient. The known sternum refractors are usually affixed in such a way that the sternum blades protrude through a skin incision and a sagittally adjoining sternum gap into the chest of the patient, while the sliding mechanism is disposed on the outside of the skin. This arrangement makes it difficult to dress the wound, in particular, with a sterile bandage.

The object of the invention is to provide a sternum refractor that in the inserted state in the patient does not protrude very much, if possible not at all, outwards beyond the surface of the skin.

### BRIEF SUMMARY OF THE INVENTION

In a preferred embodiment, a sternum spreader or retractor comprising two sternum blades, which are arranged in such a way that they can be moved in relation to each other, and each of which has a contact surface, wherein the sternum retractor comprises an elongated variable-length element with end faces, on which the sternum blades are disposed.

In another preferred embodiment, the sternum retractor as described herein, wherein the contact surfaces form two opposite end faces of an imaginary cube, inside of which the variable-length element is arranged in its entirety.

In another preferred embodiment, the sternum retractor as described herein, wherein the end faces of the variable-length element abut approximately in the center the rear side of the contact surfaces.

In another preferred embodiment, the sternum retractor as described herein, wherein the sternum blades are disposed on the variable-length element in a releasable or non-releasable manner.

In another preferred embodiment, the sternum retractor as described herein, wherein the sternum blades have a holder on their surface facing the variable-length element, and said holder has a non-circular cross section, into which the end face of the variable-length element can be inserted, in particular, in a form-fitting manner.

In another preferred embodiment, the sternum retractor as described herein, wherein the sternum blades have a pocket, which is opened toward one side and which has a cover side, on their surface facing the variable-length element; wherein said cover side has a slot, extending from the open side, and that the end faces of the variable-length element have a mating face that can be inserted into the pocket.

In another preferred embodiment, the sternum retractor as described herein, wherein the variable-length element has a central element, in which there is at least one blind hole, in which there is a thread, wherein a spacer element with a corresponding thread is screwed into the thread of the blind hole.

In another preferred embodiment, the sternum retractor as described herein, wherein the variable-length element has a central element, which has a blind hole on each of the two opposite end faces, wherein each blind hole has a thread, wherein the two threads are formed in opposite directions, and wherein a spacer element with a corresponding thread is screwed into the thread in each blind hole.

In another preferred embodiment, the sternum retractor as described herein, wherein the central element has a non-circular cross section, in particular in the form of a hexagon, in one section.

In another preferred embodiment, the sternum retractor as described herein, wherein the spacer element is disposed in a non-releasable manner on the sternum blade.

In another preferred embodiment, the sternum retractor as described herein, wherein the sternum blades are arranged parallel to each other.

In another preferred embodiment, the sternum retractor as described herein, wherein the sternum blades have in each instance a leg, which extends approximately perpendicular to the contact surface, on two opposite longitudinal edges.

### BRIEF DESCRIPTION OF THE DRAWINGS

FIG. 1a is a line drawing evidencing a view from below of a first exemplary embodiment of a sternum retractor according to the invention.

FIG. 1b is a line drawing evidencing a frontal view of the sternum retractor according to FIG. 1a.

FIG. 1c is a line drawing evidencing a lateral view of the sternum retractor according to FIG. 1a.

FIG. 1d is a line drawing evidencing a perspective view of the sternum retractor according to FIG. 1a.

FIG. 1e is a line drawing evidencing a plan view of the sternum retractor according to FIG. 1a.

FIG. 1f is a line drawing evidencing a sectional view along line A-A in FIG. 1e.

FIG. 1g is a line drawing evidencing a sectional view along line B-B in FIG. 1e.

FIG. 2a is a line drawing evidencing a view from below of a second exemplary embodiment of a sternum retractor according to the invention.

FIG. 2b is a line drawing evidencing a frontal view of the sternum retractor according to FIG. 2a.

FIG. 2c is a line drawing evidencing a lateral view of the sternum retractor according to FIG. 2a.

FIG. 2d is a line drawing evidencing a perspective view of the sternum retractor according to FIG. 2a.

FIG. 2e is a line drawing evidencing a plan view of the sternum retractor according to FIG. 2a.

FIG. 2f is a line drawing evidencing a sectional view along line A-A in FIG. 2e.

FIG. 2g is a line drawing evidencing a sectional view along line B-B in FIG. 2e.

FIG. 3a is a line drawing evidencing a view from below of a third exemplary embodiment of a sternum retractor according to the invention.

FIG. 3b is a line drawing evidencing a frontal view of the sternum retractor according to FIG. 3a.

FIG. 3c is a line drawing evidencing a lateral view of the sternum retractor according to FIG. 3a.

FIG. 3d is a line drawing evidencing a perspective view of the sternum retractor according to FIG. 3a.

FIG. 3e is a line drawing evidencing a plan view of the sternum retractor according to FIG. 3a.

FIG. 3f is a line drawing evidencing a sectional view along line A-A in FIG. 3e.

FIG. 3g is a line drawing evidencing a sectional view along line B-B in FIG. 3e.

FIG. 4a is a line drawing evidencing a view from below of a fourth exemplary embodiment of a sternum retractor according to the invention.

FIG. 4b is a line drawing evidencing a frontal view of the sternum retractor according to FIG. 4a.

FIG. 4c is a line drawing evidencing a lateral view of the sternum retractor according to FIG. 4a.

FIG. 4d is a line drawing evidencing a perspective view of the sternum retractor according to FIG. 4a.

FIG. 4e is a line drawing evidencing a plan view of the sternum retractor according to FIG. 4a.

FIG. 4f is a line drawing evidencing a sectional view along line A-A in FIG. 4e.

FIG. 4g is a line drawing evidencing a sectional view along line B-B in FIG. 4e.

FIG. 5a is a line drawing evidencing a plan view of a fifth exemplary embodiment of a sternum retractor according to the invention.

FIG. 5b is a line drawing evidencing a perspective view of the sternum retractor according to FIG. 5a.

FIG. 5c is a line drawing evidencing a sectional view along line A-A in FIG. 5a.

### DETAILED DESCRIPTION OF THE INVENTION

The terms “spreader” and “retractor” shall be used interchangeably throughout this application. The invention is a sternum spreader or refractor comprising two sternum blades, which are arranged so as to be moveable relative to each other and each of which has a contact surface, is characterized in that the sternum retractor comprises an elongated variable-length element with end faces, on which the sternum blades are disposed. According to the invention, the variable-length element is no longer bent down at an angle, but rather is designed so as to be elongated. Since the sternum blades are disposed on the end faces of the variable-length element and, as a result, are no longer transversely offset in relation of the variable-length element by means of an arm, the variable-length element is located completely between the sternum blades, so that the sternum retractor may be made as compact and flat as possible. Such an arrangement allows, in particular, the variable-length element to be arranged in the region between the sternum blades when the sternum retractor is in the inserted state in the patient. As a result, said variable-length element hardly protrudes or does not protrude beyond the skin surface to the external environment. This feature facilitates taping a sterile dressing on the wound.

According to a particularly preferred embodiment of the invention, the contact surfaces form two opposite end faces of an imaginary cube, inside of which the variable-length element is arranged in its entirety. This feature allows the sternum refractor to be made extremely flat, and it makes it possible that the sternum retractor does not protrude beyond the skin surface to the external environment, as soon as it is inserted in the chest of the patient.

It is advantageous that the end faces of the variable-length element abut the rear side of the contact surfaces approximately in the center. This feature provides a good, in particular, uniform transmission of the force to the sternum blades.

One advantageous embodiment of the invention provides that the sternum blades are releasably disposed on the variable-length element. This arrangement offers the possibility that the sternum refractor can be thoroughly cleaned. As an alternative, the sternum blades are disposed in a non-releasable manner on the variable-length element, a feature that reduces the number of components.

According to a preferred embodiment of the invention, the sternum blades have a holder on their surface facing the variable-length element; said holder has a non-circular cross section, into which the end face of the variable-length element can be inserted, in particular, in a form-fitting manner. In particular, a cut, which extends parallel to the surface of the sternum blade that faces the variable-length element, through the holder may be regarded as the cross section. A non-circular holder provides an anti-rotational lock. A form-fitting holder implements in a simple and reliable way that the sternum blades are locked in position on the variable-length element.

A particularly preferred embodiment of the invention provides that the sternum blades have a pocket having a cover side on their surface facing the variable-length element, wherein said cover side has a slot, extending from the open side, and that the end faces of the variable-length element have a mating face that can be inserted into the pocket. A pocket of this type offers the possibility of a releasable connection between the sternum blades and the variable-length element in the simplest possible way, i.e. only through insertion. In particular, this configuration offers the possibility of a releasable connection between the variable-length element and the sternum blades without having to increase the distance between the sternum blades relative to each other, in order to insert the variable-length element.

It is advantageous that the variable-length element has a central element, in which there is at least one blind hole, in which there is a thread, wherein a spacer element with a corresponding thread is screwed into the thread of the blind hole. Such a configuration of the variable-length element comprising two sub-elements, which are connected to each other by means of a thread, offers in a simple manner the possibility of a variation in length, which has a compact design.

According to a particularly preferred embodiment, the variable-length element has a central element, which has a blind hole on each of the two opposite end faces, wherein each blind hole has a thread, wherein the two threads are formed in opposite directions, and wherein a spacer element with a corresponding thread is screwed into the thread in each blind hole. Such a design of the variable-length element offers in a compact way the possibility of a variation in length of the variable-length element only if the central element is rotated relative to the two spacer elements. The use of two spacer elements offers, in particular, the possibility of a symmetrical design and a symmetrical movement of the two sternum blades toward or away from each other.

In one advantageous embodiment the spacer elements are disposed in a non-releasable manner on the corresponding sternum blades, in order to minimize the number of components.

A preferred embodiment of the invention provides that the central element has a non-circular cross section, in particular in the form of a hexagon, in one section.

This configuration offers the possibility of changing the length by means of a tool, for example, a wrench, in situ. Furthermore, such a configuration offers the possibility of a flat design of the sternum refractor, in which in the inserted state in the chest the components do not protrude beyond the surface of the skin to the external environment, since actuating elements of the sliding mechanism can be eliminated.

The sternum blades are arranged preferably parallel to each other.

It is particularly preferred that the sternum blades have in each instance a leg, which extends approximately perpendicular to the contact surface, on two opposite longitudinal edges, and which point, in particular, away from the variable-length element. These legs prevent the sternum blades from slipping off of the sternum.

### DETAILED DESCRIPTION OF THE FIGURES

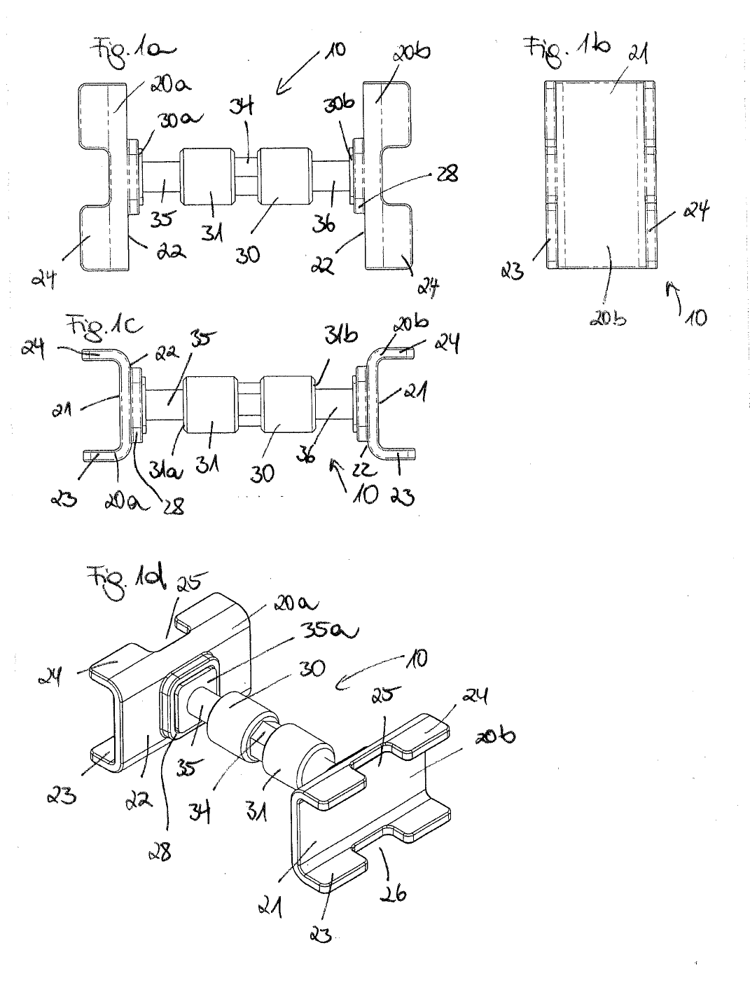

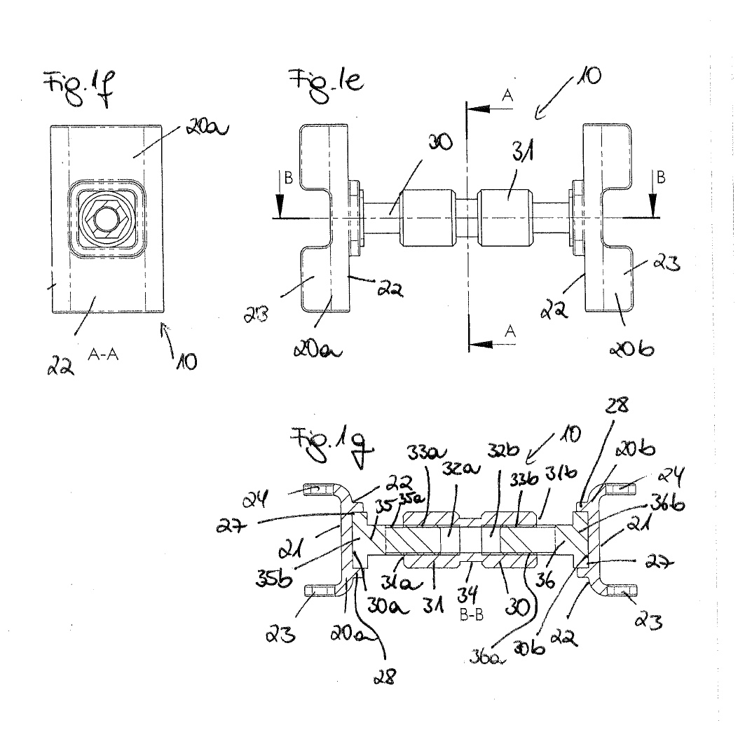

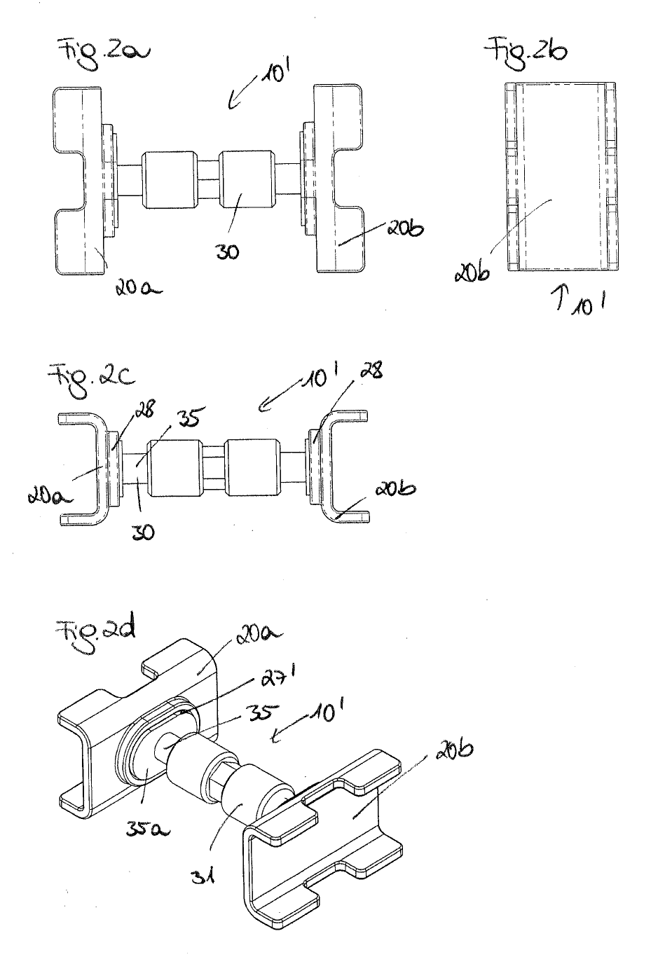

FIGS. 1a to 1g show various views of a first exemplary embodiment of a sternum refractor 10. The sternum retractor 10 has a variable-length element 30, which is designed so as to be elongated with two opposite end faces 30a, 30b. Furthermore, the sternum retractor 10 has two sternum blades 20a, 20b, which are disposed on the end faces 30a, 30b of the variable-length element 30.

Each of the sternum blades 20a, 20b has a contact surface 21 and a rear side 22 opposite the contact surface 21.

The contact surface 21 is designed to be more or less rectangular. In particular, the contact surfaces 21 of the sternum blades 20a, 20b are aligned parallel to each other. The contact surfaces 21 form, in particular, two opposite faces of an imaginary cube, within which the variable-length element 30 is disposed. In particular, a longitudinal axis of the elongated variable-length element 30 extends more or less perpendicular to the contact surfaces 21 of the sternum blades 20a, 20b.

The contact surface 21 of the sternum blades 20a, 20b can have a leg 23, 24 along two opposite longitudinal sides, in particular, on the two longer longitudinal sides. In this case each leg is aligned more or less perpendicular to the contact surface 21 and in the assembled state points away from the variable-length element 30. The legs 23, 24 improve, in particular, the abutment of the sternum blade 20a, 20b against the sternum. In one embodiment recesses 25, 26 can be arranged, in particular, approximately in the center of the legs 23, 24.

A device, which is designed for the purpose of releasably securing in an advantageous way the respective sternum blade 20a, 20b to the variable-length element 30, is arranged on the rear side 22 of the sternum blades 20a, 20b. This device is arranged, in particular, approximately in the center or in the middle of the rear side 22 of the sternum blades 20a, 20b.

In the first embodiment, which is shown in the FIGS. 1a to 1g, the fastening device has a holder 27 on the rear side 22 of the sternum blades 20a, 20b. The holder 27 is formed, in particular, by means of a web 28, which is arranged on the rear side 22, and which totally encloses in an advantageous way at least partially the holder 27. In this exemplary embodiment, the holder 27 has a cross section that is essentially rectangular, in particular square, where in this case the cross section is parallel to the rear side 22. The end faces 30a, 30b of the variable-length element 30 can be inserted into the holder 27, where they are held in a clamping, locking or form-fitting manner.

The holder 27 has a non-circular cross section in order to provide security against rotation between the sternum blade 20a, 20b and the variable-length element 30 when the sternum blades 20a, 20b are fastened to the variable-length element 30. In particular, a cut, which extends parallel to the rear face 22 of the corresponding sternum blade 20a, 20b, is considered to be the cross section.

The variable-length element 30 comprises a central element 31, which is designed, save for a non-circular section 34, in essence as a cylindrical, in particular, circularly cylindrical element. The central element 31 has two opposite end faces 31a, 31b. A blind hole 32a, 32b is arranged in at least one of the end faces 31a, 31b, in particular, in both of the end faces 31a, 31b. A thread 33a, 33b is formed in the blind hole 23a, 23b. If two blind holes 32a, 32b with threads 33a, 33b are present on the central element 31, then the threads 33a, 33b are formed advantageously in opposite directions.

A spacer element 35, 36 is inserted into the blind hole 32a, 32b. For this purpose, in particular, the spacer element 35, 36 has a section with a thread 35a, 36a, and said spacer element can be screwed into the thread 33a, 33b of the corresponding blind hole 32a, 32b. A head 35b, 36b can be formed on the spacer element 35, 36, and this head is designed for securing to the sternum blade 20a, 20b. The heads 35b, 36b of the spacer elements 35, 36 form, in particular, the end faces 30a, 30b of the variable-length element 30.

In the present embodiment the head 35b, 36b is designed, in particular, in such a way that it can be accommodated in a clamping or form-fitting manner in the holder 27. For this purpose the head 35b, 36b is formed, for example, as a flat, in essence, square element.

The non-circular section 34 of the central element 31 exhibits, in particular, the outer contour of a hexagon, so that a wrench can be applied to the central element 31. When the central element 31 is rotated, for example, by means of the wrench, the central element 31 is rotated against the spacer elements 35, 36. Since the spacer elements 35, 36 are held at a stable angle on the sternum blades 20a, 20b in the holder 27, a rotation of the central element 31 causes the spacer elements 35, 36 to be screwed, depending on the direction of rotation, both at the same time either out of the corresponding blind holes 32a, 32b or into the blind holes 32a, 32b, so that the objective of a variation in length of the variable-length element 30 is achieved.

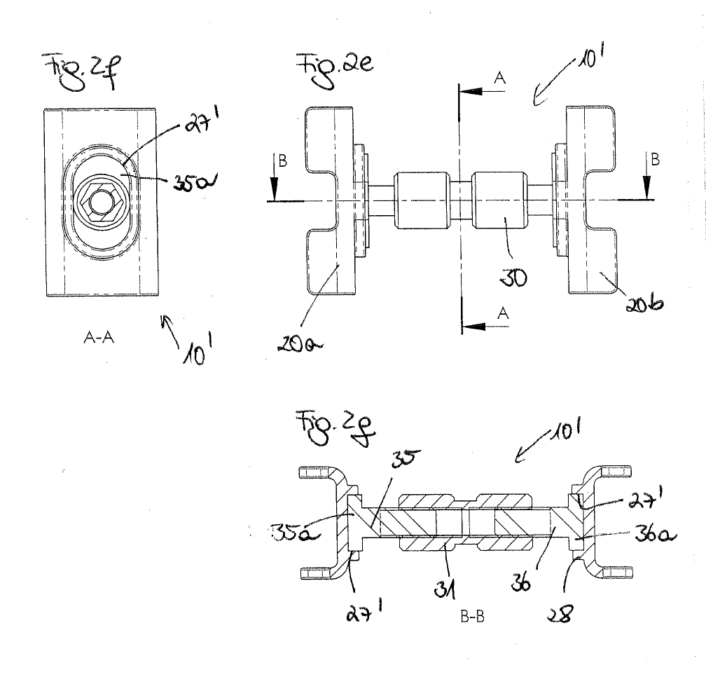

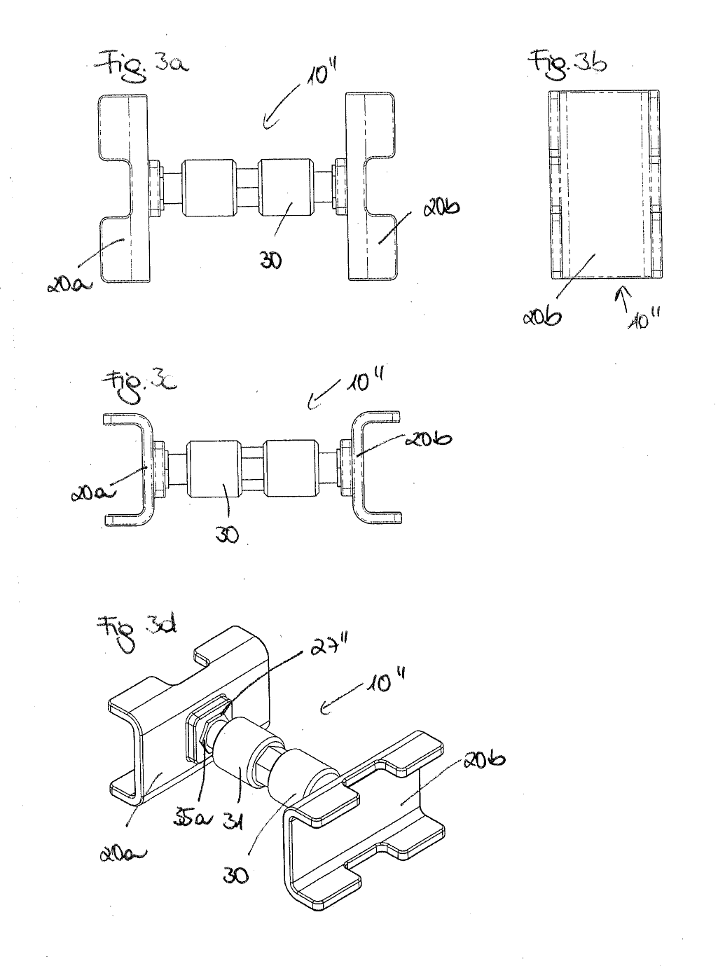

FIGS. 2a to 2g show an additional exemplary embodiment of a sternum refractor 10′ that differs from the sternum retractor 10, shown in FIGS. 1a to 1g, only in the design of the holder 27′. In the second exemplary embodiment of the sternum retractor 10′ said holder exhibits a cross section in the form of an oval, an ellipse, a rectangle with rounded corners or a rectangle with half circles or circular caps, which are attached to two opposite end faces. The heads 35b, 36b of the spacer elements 35, 36 are formed accordingly with a shape that corresponds to the shape of the recess 27′.

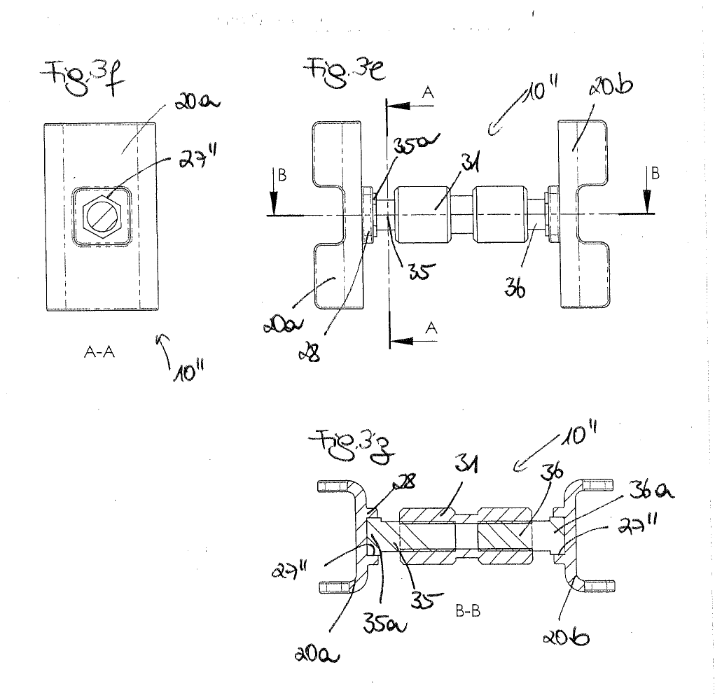

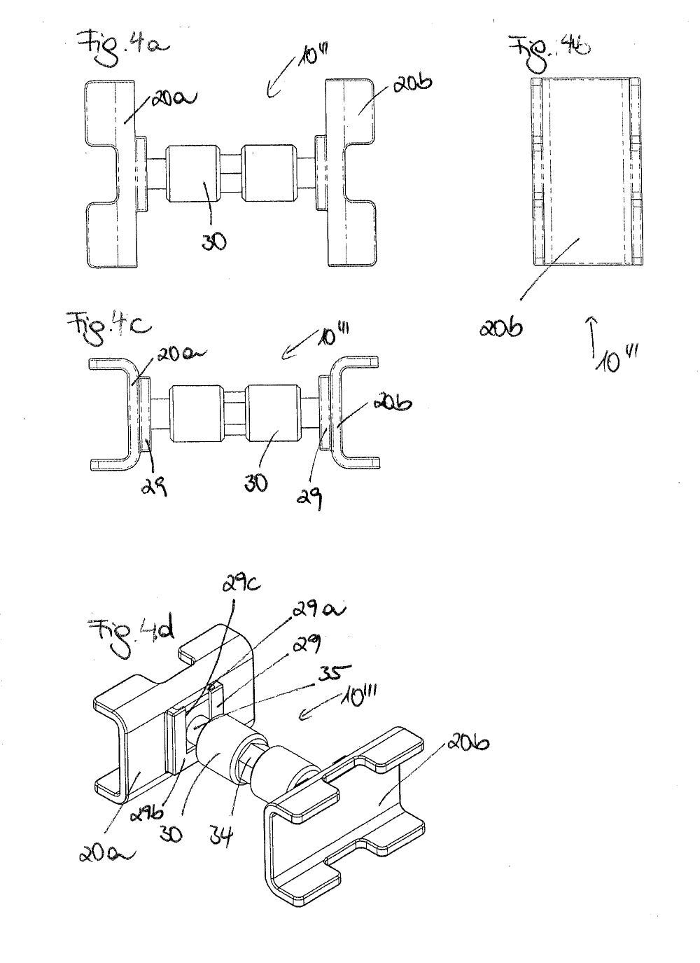

FIGS. 3a to 3g show an exemplary embodiment of a sternum refractor 10″ that differs from the sternum refractor 10, shown in FIGS. 1a to 1g, only in the design of the recess 27″. The recess 27″ has a hexagonal cross section, in which, for example, the head 35b, 36b of a screw can be inserted, so that the spacer elements 35, 36 can be formed by standard screws.

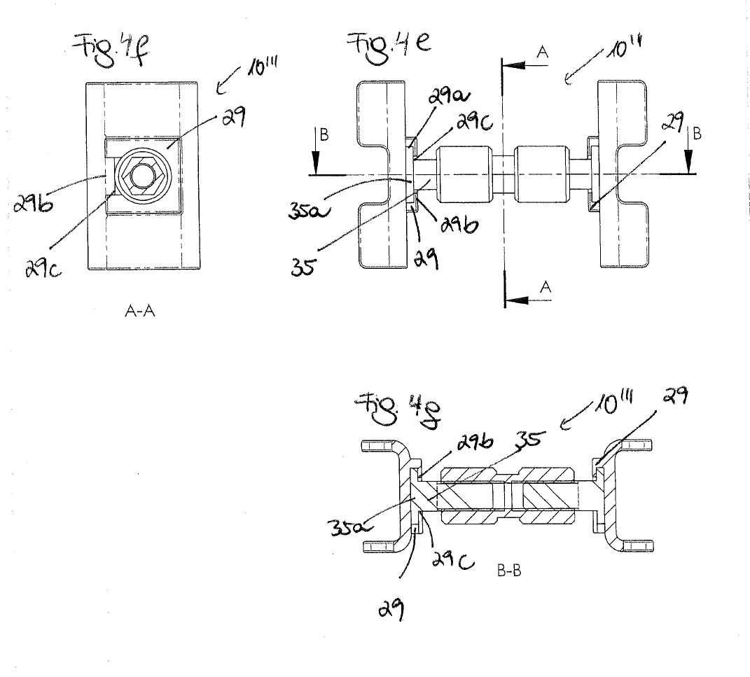

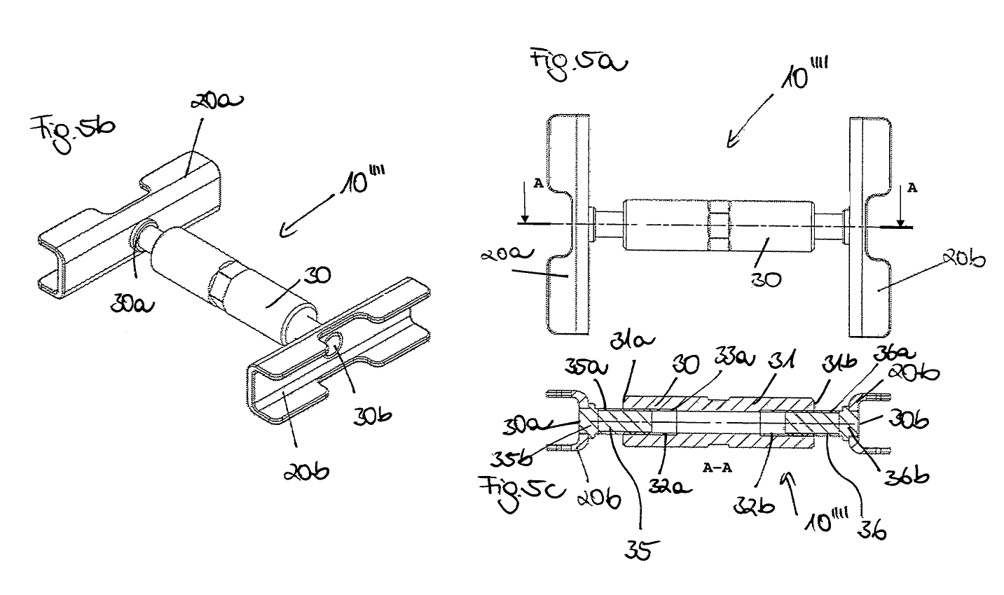

FIGS. 4a to 4g show an exemplary embodiment of a sternum retractor 10′″ that differs from the sternum retractor 10, shown in FIGS. 1a to 1g, in the design of the attachment between the variable-length element 30 and the sternum blades 20a, 20b. The rear side 22 of sternum blades 20a, 20b, according to the fourth exemplary embodiment, has a pocket 29 that is open on one side 29a. The pocket 29 has a cover side 29b, which is arranged more or less parallel to the rear side 22. The cover side 29b exhibits a slot 29c, extending from the open side 29a. The pocket 29c exhibits a non-circular cross section, which is parallel to the rear side 22, and which in the present embodiment has a rectangular or square shape. The heads 35b, 36b of the spacers 35, 36 of this exemplary embodiment have a shape that corresponds to the shape of the pocket, and are also formed, for example, rectangular or square. The heads 35b, 36b can be inserted into the pocket 29 from the open side 29a and can form in this way a mating element with the pocket 29. In this case the spacer element 35, 36 protrudes through the slot 29c beyond the pocket 29, in particular, more or less perpendicular to the rear side 22 of the sternum blade 20a, 20b. The non-circular cross section of the pockets 29 and the heads 35b, 36b, wherein the cross section of both the pockets and the heads is adjusted to each other, allows the sternum blades 20a, 20b to be secured against rotation relative to the spacer elements 35, 36.

FIGS. 5a to 5c show an exemplary embodiment of a sternum retractor 10″″ that differs from the sternum retractor 10, shown in FIGS. 1a to 1g, in the design of the attachment between the variable-length element 30 and the sternum blades 20a, 20b. In this exemplary embodiment the end faces 30a, 30b of the variable-length element 30 are rigidly secured, i.e. non-releasably, to the sternum blades 20a, 20b, for example, by soldering or welding. In particular, the head 35b, 36b of the spacer elements 35, 36 is secured, for example, by soldering or welding, to the respective sternum blade 20a, 20b. In this case the head 35b, 36b can be secured not only flush with its end face to the corresponding sternum blade 20a, 20b, but also inserted into a recess of the corresponding sternum blade 20a, 20b, where it is fixed, as shown in FIGS. 5a to 5c.

### LIST OF REFERENCE NUMBERS

10 sternum retractor
10′ sternum retractor
10″ sternum retractor
10′″ sternum retractor
10″″ sternum retractor
20a sternum blade
20b sternum blade
21 contact surface
22 rear side
23 leg
24 leg
25 recess
26 recess
27 holder
27′ holder
27″ holder
28 web
29 pocket
29a side
29b cover side
29c slot
30 variable-length element
30a end face
30b end face
31 central element
31a end face
31b end face
32a blind hole
32b blind hole
33a thread
33b thread
34 section
35 spacer element
35a thread
35b head
36 spacer element
36a thread
36b head

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable equivalents.

We claim:

1. A sternum retractor comprising two sternum blades, which are arranged in such a way that they can be moved in relation to each other, and each of which has a contact surface, wherein the sternum retractor comprises an elongated variable-length element with end faces, on which the sternum blades are disposed.

2. The sternum retractor of claim 1, wherein the contact surfaces form two opposite end faces of an imaginary cube, inside of which the variable-length element is arranged in its entirety.

3. The sternum retractor of claim 1, wherein the end faces of the variable-length element abut approximately in the center the rear side of the contact surfaces.

4. The sternum retractor of claim 1, wherein the sternum blades are disposed on the variable-length element in a releasable or non-releasable manner.

5. The sternum retractor of claim 1, wherein the sternum blades have a holder on their surface facing the variable-length element, and said holder has a non-circular cross section, into which the end face of the variable-length element can be inserted, in particular, in a form-fitting manner.

6. The sternum retractor of claim 1, wherein the sternum blades have a pocket, which is opened toward one side and which has a cover side, on their surface facing the variable-length element; wherein said cover side has a slot, extending from the open side, and that the end faces of the variable-length element have a mating face that can be inserted into the pocket.

7. The sternum retractor of claim 1, wherein the variable-length element has a central element, in which there is at least one blind hole, in which there is a thread, wherein a spacer element with a corresponding thread is screwed into the thread of the blind hole.

8. The sternum retractor of claim 1, wherein the variable-length element has a central element, which has a blind hole on each of the two opposite end faces, wherein each blind hole has a thread, wherein the two threads are formed in opposite directions, and wherein a spacer element with a corresponding thread is screwed into the thread in each blind hole.

9. The sternum retractor of claim 7, wherein the central element has a non-circular cross section, in particular in the form of a hexagon, in one section.

10. The sternum retractor of claim 7, wherein the spacer element is disposed in a non-releasable manner on the sternum blade.

11. The sternum retractor of claim 1, wherein the sternum blades are arranged parallel to each other.

12. The sternum retractor of claim 1, wherein the sternum blades have in each instance a leg, which extends approximately perpendicular to the contact surface, on two opposite longitudinal edges.
